(19) United States
(12) Patent Application Publication
(10) Pub. No.: US 2004/0220102 A1
(43) Pub. Date: Nov. 4, 2004

(54) Method of treating disc herniation and disc degeneration with concentrated growth and differentiation factors

(76) Inventor: Bret A. Ferree, Cincinnati, OH (US)

Correspondence Address: John G. Posa, Gifford, Krass, Groh, Sprinkle,, Anderson & Citkowski, P.C., 280 N. Old Woodward Ave., Suite 400, Birmingham, MI, 48009-5394, US

(21) Appl. No.: 10/853,443

(22) Filed: May 25, 2004

Related U.S. Application Data
(63) Continuation of application No. 09/897,000, filed on Jul. 2, 2001.
(60) Provisional application No. 60/215,445, filed on Jun. 30, 2000.

(51) Int. Cl. A61K 38/18

(52) U.S. Cl. 514/12

(57) ABSTRACT

Soluble regulators such as growth factors and differentiation factors are used to treat disc disease and herniation. Such substances may be produced with recombinant genetic techniques, or obtained from animal sources. In the preferred embodiment the materials are concentrated from a patient's blood then injected into the epidural space of the spinal canal and or the intervertebral disc using techniques well known to those skilled in the art. The blood is centrifuged to obtain platelets, and the platelets release the soluble regulators/growth factors by adding a mixture of calcium chloride and topical bovine thrombin. According to one example, 6 ml of platelet rich plasma is combined with 1 ml of the calcium chloride—thrombin mixture and injected into the disc or spinal canal. Alternatively, the platelet rich plasma and calcium chloride—thrombin mixture may be injected separately. Soluble regulators obtained from other sources or different amounts of the platelet rich plasma than described above could also be used.

### REFERENCE TO RELATED APPLICATION

This application claims priority from U.S. provisional application Ser. No. 60/215,445, filed Jun. 30, 2000, the entire contents of which is incorporated herein by reference.

### FIELD OF THE INVENTION

This method relates generally to treatment of disc herniation or disc degeneration

### BACKGROUND OF THE INVENTION

Eighty-five percent of the population will experience low back pain at some point. Fortunately, the majority of people recover from their back pain with a combination of benign neglect, rest, exercise, medication, physical therapy, or chiropractic care. A small percent of the population will suffer chronic low back pain. The cost of treatment of patients with spinal disorders plus the patient's lost productivity is estimated at 25 to 100 billion dollars annually.

Seven cervical (neck), 12 thoracic, and 5 lumbar (low back) vertebrae form the normal human spine. Intervertebral discs reside between adjacent vertebra with two exceptions. First, the articulation between the first two cervical vertebrae does not contain a disc. Second, a disc lies between the last lumbar vertebra and the sacrum (a portion of the pelvis).

The spine supports the body, and protects the spinal cord and nerves. The vertebrae of the spine are also supported by ligaments, tendons, and muscles which allow movement (flexion, extension, lateral bending, and rotation). Motion between vertebrae occurs through the disc and two facet joints. The disc lies in the front or anterior portion of the spine. The facet joints lie laterally on either side of the posterior portion of the spine.

The human intervertebral disc is an oval to kidney bean shaped structure of variable size depending on the location in the spine. The outer portion of the disc is known as the annulus fibrosis. The annulus is formed of 10 to 60 fibrous bands. The fibers in the bands alternate their direction of orientation by 30 degrees between each band. The orientation serves to control vertebral motion (one half of the bands tighten to check motion when the vertebra above or below the disc are turned in either direction).

The annulus contains the nucleus. The nucleus pulpous serves to transmit and dampen axial loads. A high water content (70-80 percent) assists the nucleus in this function. The water content has a diurnal variation. The nucleus imbibes water while a person lies recumbent. Activity squeezes fluid from the disc. Nuclear material removed from the body and placed into water will imbibe water swelling to several times its normal size. The nucleus comprises roughly 50 percent of the entire disc. The nucleus contains cells (chondrocytes and fibrocytes) and proteoglycans (chondroitin sulfate and keratin sulfate). The cell density in the nucleus is on the order of 4,000 cells per micro liter.

Interestingly, the adult disc is the largest avascular structure in the human body. Given the lack of vascularity, the nucleus is not exposed to the body's immune system. Most cells in the nucleus obtain their nutrition and fluid exchange through diffusion from small blood vessels in adjacent vertebra.

The disc changes with aging. As a person ages the water content of the disc falls from approximately 85 percent at birth to 70 percent in the elderly. The ratio of chondroitin sulfate to keratin sulfate decreases with age. The ratio of chondroitin 6 sulfate to chondroitin 4 sulfate increases with age. The distinction between the annulus and the nucleus decreases with age. These changes are known as disc degeneration. Generally disc degeneration is painless.

Premature or accelerated disc degeneration is known as degenerative disc disease. A large portion of patients suffering from chronic low back pain are thought to have this condition. As the disc degenerates, the nucleus and annulus functions are compromised. The nucleus becomes thinner and less able to handle compression loads. The annulus fibers become redundant as the nucleus shrinks. The redundant annular fibers are less effective in controlling vertebral motion. The disc pathology can result in: 1) bulging of the annulus into the spinal cord or nerves; 2) narrowing of the space between the vertebra where the nerves exit; 3) tears of the annulus as abnormal loads are transmitted to the annulus and the annulus is subjected to excessive motion between vertebra; and 4) disc herniation or extrusion of the nucleus through complete annular tears.

Current surgical treatments of disc degeneration are destructive. One group of procedures removes the nucleus or a portion of the nucleus; lumbar discectomy falls in this category. A second group of procedures destroy nuclear material; Chymopapin (an enzyme) injection, laser discectomy, and thermal therapy (heat treatment to denature proteins) fall in this category. A third group, spinal fusion procedures either remove the disc or the disc's function by connecting two or more vertebra together with bone. These destructive procedures lead to acceleration of disc degeneration. The first two groups of procedures compromise the treated disc. Fusion procedures transmit additional stress to the adjacent discs. The additional stress results in premature disc degeneration of the adjacent discs.

Prosthetic disc replacement offers many advantages. The prosthetic disc attempts to eliminate a patient's pain while preserving the disc's function. Current prosthetic disc implants, however, either replace the nucleus or the nucleus and the annulus. Both types of current procedures remove the degenerated disc component to allow room for the prosthetic component.

Several hundred thousand patients undergo disc operations each year. Approximately five percent of these patients will suffer recurrent disc herniation, which results from a void or defect which remains in the outer layer (annulus fibrosis) of the disc after surgery involving partial discectomy. The defect acts as a pathway for additional material to protrude into the nerve, resulting in the recurrence of the herniation. This results in pain and further complications, in many cases.

Apart from destructive techniques, patients with herniated intervertebral discs and degenerative disc disease conservatively be treated by rest, physical therapy, oral medication, and chiropractic care. Patients that do not respond to conservative care generally undergo an injection of steroids into the epidural space of their spinal canal (epidural space) or surgery. Steroid injection reduces the inflammation surrounding herniated or degenerated discs. Decreased inflammation may reduce the pain from the disc. Unfortunately, steroid injection may hinder the healing process. Although growth factors and differentiation factors (soluble regulators) induce the healing process, it is believed that steroids may interfere with the cascade of these healing factors normally found in the body.

Given the large number of patients each year which require surgery to treat disc disease and herniation, with substantial implications in terms of the cost of medical treatment and human suffering, any solution to improve the effectiveness of non-surgical treatments would be welcomed by the medical community.

### SUMMARY OF THE INVENTION

Broadly, this invention takes advantage of soluble regulators such as growth factors and differentiation factors to treat disc disease and herniation. Such substances may be produced with recombinant genetic techniques, or obtained from animal sources. In the preferred embodiment, the materials are concentrated from a patient's blood and injected into the epidural space of the spinal canal and or the intervertebral disc using techniques well known to those skilled in the art.

The blood is centrifuged to obtain platelets, and the platelets release the soluble regulators/growth factors by adding a mixture of calcium chloride and topical bovine thrombin. According to one example, 6 ml of platelet rich plasma is combined with 1 ml of the calcium chloride—thrombin mixture and injected into the disc or spinal canal. Alternatively, the platelet rich plasma and calcium chloride—thrombin mixture may be injected separately. Soluble regulators obtained from other sources or different amounts of the platelet rich plasma than described above could also be used.

### DETAILED DESCRIPTION OF THE INVENTION

This invention recognizes that soluble regulators in the form of growth factors and differentiation factors may be used to treat disc disease and herniation nonsurgically. A list of useful substances would include at least the following: TGF-α, β1, -2; EGF, IGF-I; PDGF; FGF; IL-I, -1a, -1b, -2, -3, -4, -5, -6, . . . n; BMP-1, -2, -3, -4, -5, -6, -7, -8, -8B, -9, -12, -13, . . . n; VEGF; and recombinant forms thereof.

In accordance with the invention, such substances may be concentrated from a patient's blood, produced with recombinant genetic techniques, or obtained from animal sources. The soluble regulators are injected into the epidural space of the spinal canal and or the intervertebral disc using techniques well known to those skilled in the art.

For example, many of the factors can be obtained from the platelets from a patient's blood. Approximately 400-500 ml of blood is withdrawn from a patient using standard techniques. The blood is centrifuged with standard cell sorting equipment such as that sold by Cobe Cardiovascular Inc. of Arvada, Colo. Centrifugation separates the blood into platelet poor plasma, platelet rich plasma, and red blood cells. The platelet poor plasma and red blood cells are returned to the patient intravenously. The platelets are forced to release the soluble regulators/growth factors by adding a mixture of 10 ml of 10% calcium chloride and 10,000 units of topical bovine thrombin (Gentrac).

For example, 6 ml of platelet rich plasma would be combined with 1 ml of the calcium chloride—thrombin mixture and injected into the disc or spinal canal. Alternatively, the platelet rich plasma and calcium chloride—thrombin mixture may be injected separately. Soluble regulators obtained from other sources or different amounts of the platelet rich plasma than described above could also be used.

I claim:

1.-4. (Canceled)

5. A treatment procedure for disc herniation or degenerative disc disease in a patient, including the step of:
delivering the bone morphogenic protein BMP-7 into the central spinal canal or disc as part of the treatment procedure.

6. The method of claim 5, further including the steps of:
withdrawing a volume of blood from the patient; and
obtaining the BMP-7 from the volume of blood.

7. The method of claim 5, including the step of:
obtaining the BMP-7 from recombinant genetic techniques or animal sources.

8. The method of claim 5, wherein the BMP-7 is injected into the spinal canal or disc.
